SEQUENCE LISTING (6 sequences)

SEQ ID NO: 1 - DNA, 16 bp, Artificial Sequence
  Description of Artificial Sequence adaptor
atcccggacgaaggcc

SEQ ID NO: 2 - DNA, 13 bp, Artificial Sequence
  Description of Artificial Sequence adaptor
ggccttcgtccgg

SEQ ID NO: 3 - DNA, 1134 bp, Saccharomyces cerevisiae
atgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgccggaatttatggccatggaggccccggggatccgaaggaacgcaagagccatgtcacaaaaggataacctactcgacaatccggttgaatttttaaaagaggtcagagaaagttttgatattcagcaagatgttgatgccatgaaaagaatccgacacgatcttgatgttataaaagaggaaagcgaagcaagaattagtaaagagcattcaaaggtttctgagtcgaacaagaaattgaatgcggaaagaataaatgttgctaaattggagggagacttagaatatactaacgaagagagcaatgagtttggtagtaaagacgaactagttaaacttctgaaagatttggacggattggaacgtaatattgtgtcacttcgaagtgaattggacgaaaagatgaaattgtacctcaaagatagtgaaataatatccacaccgaacggttccaaaataaaagcaaaagtaattgaacctgagctggaagaacaaagtgcggtcaccccggaagcaaacgaaaatattctaaaattgaagctatacagatctttaggagttattttggatttagaaaatgatcaagtccttattaacagaaaaaatgatgggaatattgatattttacccttggacaataacctcagcgatttctataagaccaaatacatctgggaaagattaggaaagtga

SEQ ID NO: 4 - protein, 378 aa, Saccharomyces cerevisiae
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSPEFMAMEAPGIRRNARAMSQKDNLLDNPVEFLKEVRESFDIQQDVDAMKRIRHDLDVIKEESEARISKEHSKVSESNKKLNAERINVAKLEGDLEYTNEESNEFGSKDELVKLLKDLDGLERNIVSLRSELDEKMKLYLKDSEIISTPNGSKIKAKVIEPELEEQSAVTPEANENILKLKLYRSLGVILDLENDQVLINRKNDGNIDILPLDNNLSDFYKTKYIWERLGKZ

SEQ ID NO: 5 - DNA, 47 bp, Saccharomyces cerevisiae
gaattgtaatacgactcactatagggaggtgatgaagataccccacc

SEQ ID NO: 6 - DNA, 54 bp, Saccharomyces cerevisiae
agatgcaattaaccctcactaaagggagacggggtttttcagtatctacgattc

(19) United States
(12) Patent Application Publication
(10) Pub. No.: US 2002/0025552 A1
(43) Pub. Date: Feb. 28, 2002

(54) SCREENING INTERACTOR MOLECULES WITH WHOLE GENOME OLIGONUCLEOTIDE OR POLYNUCLEOTIDE ARRAYS

(75) Inventors: PIERRE LEGRAIN, PARIS (FR); MICHELINE FROMONT-RACINE, VILLEJUIF (FR)

Correspondence Address: FINNEGAN HENDERSON FARABOW, GARRETT & DUNNER, 1300 I STREET NW, WASHINGTON, DC, 20005

(73) Assignee: Institut Pasteur

(21) Appl. No.: 09/154,972

(22) Filed: Sep. 17, 1998

Related U.S. Application Data
(63) Continuation-in-part of application No. 09/003,335, filed on Jan. 6, 1998.

(51) Int. Cl. C12P 21/06; C12P 19/34; G01N 33/564; A61K 31/70

(52) U.S. Cl. 435/69.1; 514/44; 435/91.1; 436/508

(57) ABSTRACT

This invention relates to methods for the identification of nucleic acids by direct hybridization to high-density oligonucleotide arrays. The methods of this invention comprise the steps of: (1) screening a DNA library, such as an S. cerevisiae genomic DNA library, by performing a double hybrid screening method with a recombinant vector containing a DNA insert encoding a candidate protein of interest and then selecting the clones from the DNA library that code for proteins that interact with the candidate protein of interest; and (2) hybridizing the DNA inserts contained in the clones that have been selected in step (1) using an oligonucleotide probe matrix wherein the probe locations on the host genome cover all of the coding sequences, determining the hybridization location and consequently, the gene coding for a specific protein that interacts with the candidate protein of interest in the double hybrid screening system. This invention is also directed to the polynucleotides obtained by the methods of this invention, the polypeptides encoded by those polynucleotides and the DNA arrays utilized in the methods of this invention.

This application is a continuation-in-part of application Ser. No. 09/003,335, filed Jan. 6, 1998.

This invention is directed to methods for the identification of nucleic acids by direct hybridization to high-density oligonucleotide arrays, to the nucleic acids identified by these methods, and to the oligonucleotide arrays. The methods of this invention are applicable to the analysis of a wide range of genetic selections with outputs of high complexity.

The work underlying this invention was supported by an NIH Institutional Training Grant in Genome Science. The government may have certain rights in this invention.

### BACKGROUND OF THE INVENTION

An estimated 6,000 genes were identified upon the completion of sequencing the Saccharomyces cerevisiae genome. Fewer than half of these genes have a known biological function.^{1,2} Understanding how these newly sequenced genes function in both defined and emerging biochemical pathways is a major challenge for researchers in the post-genome era. Efficient functional characterization of these genes requires strategies for scaling genetic analyses to the whole genome level.³ Determination of MRNA gene expression patterns, disruption phenotypes, and protein-protein interactions are key questions, which need to be addressed for every gene in a genome.

Plasmid-based library selections are an established approach to the functional analysis of uncharacterized genes, and can help elucidate biological function by identifying, for example, physical interactors for a gene and genetic enhancers and suppressors of mutant phenotypes. However, the application of these selections to every gene in a eukaryotic genome involves the need to manipulate and sequence hundreds of DNA plasmids. Thus, applying traditional methods of functional analysis to every gene in a genome is limited by labor and cost.

Because the discovery of thousands of uncharacterized genes by genome sequencing projects has increased the need for methods of large scale functional analysis, several approaches have been initiated to identify genes that, when disrupted or removed, lead to selective growth disadvantages.¹⁴⁻¹⁶ A promising complementary approach is the application of established genetic screens to every gene in an organism in an attempt to assign a biological function to every open reading frame. Genome-wide analyses based on two-hybrid screens, enhanced synthetic lethal screens, and screens for signal peptide sequences have been proposed.¹⁷⁻¹⁹

The two hybrid assay exploits the ability of a pair of interacting proteins to bring a transcription activation domain into close proximity with a DNA-binding site that regulates the expression of an adjacent reporter gene. The assay employs chimeric genes which express two types of hybrid proteins. The second hybrid contains the DNA binding domain of a transcriptional activator fused to a second test protein. The first hybrid protein contains a transcriptional activation domain fused to a first test protein. If the two test proteins are able to interact, they bring the two domains of the transcriptional activator into close proximity sufficient to cause transcription, which can then be detected by the activity of a marker gene that contains a binding site for the DNA-binding domain.

The two-hybrid assay can be used to test a multiplicity of proteins simultaneously to determine whether they interact with a known protein. For example, a DNA fragment encoding the DNA-binding domain may be fused to a DNA fragment encoding the known protein in order to provide one hybrid. This hybrid is introduced into the cells carrying a marker gene. For the first hybrid, a library of plasmids can be constructed which may include, for example, total mammalian cDNA fused to the DNA sequence encoding the activation domain. This library is introduced into the cells carrying the second hybrid. If any individual plasmid from the library encodes a protein that is capable of interacting with the known protein, a positive signal will be obtained. However, because repetitive dideoxy sequencing is required to exhaustively identify the results of a screen, application of these methods to tens of thousands of genes is also limited by time, labor, and expense.

Two-hybrid screens for protein-protein interactions provide a genetic tool that can be applied, in principle, to every gene in a genome. The Escherichia coli bacteriophage T7 genome has already been characterized with exhaustive two-hybrid screening and sequencing for each known gene. Even with the use of novel strategies for highly efficient two-hybrid screening, however, an analysis of all genes encoded in the human genome would require sequencing of approximately 1×10⁶ sequence fragments. As an alternative, genes may be individually cloned into two-hybrid vectors and tested in a pairwise manner. One disadvantage of this approach is that testing only the fall length form of a gene might fail to identify those interactions that occur only with isolated domains of a protein.²⁰ Functional selections that need to be performed in mammalian cells would also benefit from more highly parallel analysis. For example, it is conceivable to select for human genes that yield phenotypes, such as increased drug or pathogen resistance, when overexpressed in cell lines. The use of array hybridization to analyze results from these screens would eliminate the need to maintain large numbers of individual clones in tissue culture until they can be sequenced. Thus, the present invention overcomes the problems associated with the prior art through the use of DNA arrays or matrices, permitting highly parallel identification of the sequence and orientation of nucleic acid elements in a pool.

### SUMMARY OF THE INVENTION

The methods of this invention comprise the steps of: (1) screening a DNA library, such as an S. cerevisiae genomic DNA library, by performing a double hybrid method with a recombinant vector containing a DNA insert encoding a candidate protein of interest and then selecting the clones from the DNA library that code for proteins that interact with the candidate protein of interest; and (2) hybridizing the DNA inserts contained in the clones that have been selected in step (1) using an oligonucleotide probe matrix, wherein the probe locations on the host genome cover all of the coding sequences, determining the hybridization location and consequently, the gene coding for a specific protein that interacts with the candidate protein of interest in the double hybrid screening system. Thus, the methods of this invention allow screening at a very large scale for DNA sequences having functional utility and avoid the systematic sequencing of the DNA inserts of interest required by prior art methods.

This invention is also directed to the polynucleotides obtained by the methods of this invention and the polypeptides encoded by those polynucleotides. In addition, the invention is directed to the DNA arrays or matrices utilized in the methods of this invention.

Oligonucleotide arrays can be synthesized for any organism for which complete or partial sequence information is available. The time required to analyze the results of a genetic selection can be drastically reduced, making it feasible to apply conventional screens to very large numbers of genes in a mammalian genome. Analysis of screens by array hybridization is adaptable to any genome-wide functional selection or experiment where the output is a set of nucleic acid sequences.

For example, DNA arrays containing oligonucleotides complementary to every gene in the Saccharomyces cerevisiae genome can be used to analyze the results from plasmid based genetic screens in a single experiment. Based on the recently completed sequence of Saccharomyces cerevisiae, the first high density arrays containing oligonucleotides complementary to every gene in the yeast genome have been designed and synthesized. Two-hybrid protein-protein interaction screens were carried out for Saccharomyces cerevisiae genes implicated in mRNA splicing and microtubule assembly. Hybridization of labeled DNA derived from positive clones is sufficient to characterize the results of a screen in a single experiment allowing rapid detection of both established and novel biological interactions. These results demonstrate the use of oligonucleotide arrays for the analysis of two-hybrid screens. This approach is generally applicable to the analysis of a range of genetic selections with outputs of high complexity.

### BRIEF DESCRIPTION OF THE DRAWINGS

FIG. 1 represents a method for identifying sequences following a genetic selection. Rather than individual purification and dideoxysequencing, all clones are pooled from plates, and plasmid DNA is isolated in a single purification. PCR amplification using primers with 3′ sequence corresponding to the vector sequence is used to selectively enrich for insert DNA from the plasmid pool. Amplified insert DNA is fragmented with DNAse I, labeled with biotin-ddATP, and hybridized to an array containing oligonucleotide probes for every gene in the yeast

FIG. 2 depicts fluorescence images of a high-density oligonucleotide array containing 25-mer probes for nearly every gene on Saccharomyces cerevisiae chromosomes 5 through 10.

FIG. 2a depicts the fluorescence pattern obtained following hybridization of 11 control genes: YEL002c, YEL003w, YEL005c, YEL006w, YEL018w, YEL019c, YEL021w, YEL024w, YHL014c, YHL045w, and YHL044c. Dark areas correspond to probes for genes not present in the control pool. FIG. 2b provides a close-up view of gene YHL014c, which show the exact probe features that hybridize to the insert. Red grid highlights all probe features for YHL014c. The top row of probe elements contain oligonucleotides perfectly complementary to gene sequence, while bottom rows contain a mismatch in the central position of the oligonucleotide. Approximate locations of complementary oligonucleotide probes along the YHL014c ORF are also shown.

FIG. 3 depicts a fluorescence image of a portion of a high-density oligonucleotide array containing 25-mer probes to nearly every gene on Saccharomyces cerevisiae chromosomes 5 through 10 following hybridization of YMR117c two-hybrid sample. The three lighted strips correspond to probes covering nucleotides 156-654 of ORF YER018c, nucleotides 1860-2484 of YER032w, and nucleotides 4092-4452 of YGL197w. Terminal probes are described as the most 5′ nucleotide of the most 5′ probe and the most 3′ nucleotide of the most 3′ probes that gave a positive signal. Dark areas correspond to probes for genes not present following genetic selection.

### DETAILED DESCRIPTION OF THE INVENTION

The present invention provides methods for screening polynucleotides, such as polynucleotides contained in the genome or in a cDNA obtained from the MRNA of a given prokaryotic or eukaryotic host or in a DNA insert of a random peptide DNA library. In essence, the methods of this invention comprise the steps of: (1) subjecting the polynucleotide of interest to a two-hybrid screening method; and (2) subjecting the polynucleotides selected at step (1) to a hybridization reaction onto a matrix substrate onto which oligonucleotide or polynucleotide probes have been immobilized (i.e., DNA array).

Any two-hybrid screening method may be used to complete step (1) of the methods of +this invention. For example, the yeast two hybrid system developed by Fields and coworkers²¹ utilizes hybrid genes to detect protein-protein interactions by means of direct activation of a reporter-gene expression. U.S. Pat. Nos. 5,283,173 and 5,468,614 describing this technique are relied upon and incorporated by reference. Mammalian two hybrid systems using β-galactosidase complementation to monitor protein-protein interactions in intact eukaryotic cells,^{22,23} phage display,²⁴ and double tagging assays²⁵ represent alternative two-hybrid assay approaches to screen complex libraries of proteins for direct interaction with a given ligand. In addition, reverse two hybrid screening procedures, such as those described by White²⁶ and Vidal et al.,^{27,28} can be utilized in the methods of this invention. Most preferably, the two-hybrid system utilized in the methods of this invention is that described by Daniel Ladant et al. in U.S. provisional patent application No. 60/067,308 entitled A BACTERIAL MULTI-HYBRID SYSTEM AND APPLICATIONS THEREOF, filed Dec. 4, 1997, the entire disclosure of which is relied upon and incorporated herein by reference.

The preparation and use of high density DNA arrays has been described in International patent applications WO 97/29212, WO 97/27317, WO 97/10365, and WO 92/10588, the disclosures of which are relied upon and incorporated herein by reference. See also, Wodicka, L. et al. (1997) Nature Biotechnology. 15, 1359-1367.

One embodiment of this invention (designated “Method 1” for convenience) provides a method for selecting a polynucleotide encoding a first polypeptide that is able to interact with a second polypeptide of interest. Specifically, this method comprises the following steps:

a) providing a recombinant host cell containing a detectable gene, wherein the detectable gene expresses a detectable polypeptide when the detectable gene is activated by an amino acid sequence including a transcriptional activation domain, such as the transcription activation domain of the GAL4 protein;

b) providing a first chimeric gene that is capable of being expressed in the host cell, the first chimeric gene comprising a DNA sequence that encodes a first hybrid polypeptide encoded by a given prokaryotic or eukaryotic organism, said first hybrid polypeptide comprising:

(i) the transcriptional activation domain; and

(ii) a first test polypeptide that is to be tested for interaction with the second test polypeptide;

c) providing a second chimeric gene that is capable of being expressed in the host cell, the second chimeric gene comprising a DNA sequence that encodes a second hybrid polypeptide, the second hybrid polypeptide comprising:

(i) a DNA-binding domain, e.g., the DNA binding domain of the GAL4 protein, that recognizes a binding site on the detectable gene in the host cell; and

(ii) a second test polypeptide that is to be tested for interaction with at least one first test polypeptide;

wherein interaction between the first test polypeptide and the second test polypeptide in the host cell causes the transcriptional activation domain to activate transcription of the detectable gene;

d) introducing the first chimeric gene and the second chimeric gene into the host cell;

e) subjecting the host cell to conditions under which the first hybrid polypeptide and the second hybrid polypeptide are expressed in sufficient quantity for the detectable gene to be activated;

f) selecting the host cell clones for which the detectable gene has been expressed to a degree greater than expression in the absence of interaction between the first test polypeptide and the second test polypeptide;

g) optionally pooling the clones that have been positively selected at step f)

h) amplifying the polynucleotides of interest contained in the clones of step f) or g) with a pair of oligonucleotide primers respectively hybridizing with a plasmid sequence located at the 5′ end of the polynucleotide of interest and with a sequence complementary to a plasmid sequence located at the 3′ end of the polynucleotide of interest coding for the first polypeptide;

i) hybridizing the amplified polynucleotides obtained at step h) to a matrix substrate on which has been bound, at known locations, a plurality of sets of oligonucleotide or polynucleotide probes of predetermined sequence, each bound set of oligonucleotide or polynucleotide probes being able to hybridize with a specific polynucleotide carried by the genome of the organism from which the polynucleotide coding for the first test polypeptide belongs;

j) detecting the locations of the polynucleotide hybrid complexes obtained at step i) on the matrix substrate; and

k) optionally determining the quantity of each hybrid complex detected at step j).

Most preferably, the second chimeric gene is provided to the recombinant cell host before the introduction of the first chimeric gene.

An alternate embodiment of the invention (designated “Method 2” for convenience) provides a method for selecting a polynucleotide encoding a first polypeptide that inhibits the interaction between a second polypeptide and a third polypeptide. Specifically, this method comprises the following steps:

a) providing a recombinant host cell containing a detectable gene, wherein the detectable gene expresses a detectable polypeptide when the detectable gene is activated by an amino acid sequence including a transcriptional activation domain, e.g., GAL4;

b) providing a first gene that is capable of being expressed in the host cell, said first gene comprising a DNA sequence that encodes a first polypeptide encoded by a given prokaryotic or eukaryotic organism, and for which its inhibition property on the interaction between a second and a third polypeptide is tested;

c) providing a second chimeric gene that is capable of being expressed in host cell, the second chimeric gene comprising a DNA sequence that encodes a second hybrid polypeptide encoded by a given prokaryotic or eukaryotic organism, said second hybrid polypeptide comprising:

(i) the transcriptional activation domain; and

(ii) a second test polypeptide that interacts with a third polypeptide;

d) providing a third chimeric gene that is capable of being expressed in the host cell, the third chimeric gene comprising a DNA sequence that encodes a third hybrid polypeptide, the third hybrid polypeptide comprising:

(i) a DNA-binding domain, such as GAL4, that recognizes a binding site on the detectable gene in the host cell; and

(ii) a third test polypeptide that interacts with the second test polypeptide;

wherein interaction between the second test polypeptide and the third test polypeptide in the host cell causes the transcriptional activation domain to activate transcription of the detectable gene;

e) introducing the first gene, the second chimeric gene, and the third chimeric gene into the host cell;

f) subjecting the host cell to conditions under which the second hybrid polypeptide and the third polypeptide are expressed in sufficient quantity for the detectable gene to be activated;

g) selecting the host cell clones for which the detectable gene has been expressed to a degree lesser than its expression level in the absence of expression of the first polypeptide;

h) optionally pooling the clones that have been positively selected at step g);

i) amplifying the polynucleotides of interest contained in the clones of step g) or h) with a pair of oligonucleotide primers respectively hybridizing with a plasmid sequence located at the 5′ end of the polynucleotide of interest and with a sequence complementary to a plasmid sequence located at the 3′ end of the polynucleotide of interest coding for the first polypeptide;

j) hybridizing the amplified polynucleotides obtained at step i) to a matrix substrate on which has been bound, at known locations, a plurality of sets of oligonucleotide or polynucleotide probes of predetermined sequence, each bound set of oligonucleotide or polynucleotide probes being able to hybridize with a specific polynucleotide carried by the genome of the organism from which the polynucleotide coding for the first test polypeptide belongs;

k) detecting the locations of the polynucleotide hybrid complexes obtained at step j) on the matrix substrate;

l) optionally determining the quantity of each hybrid complex detected at step i).

Most preferably, the second and the third chimeric genes are provided to the recombinant cell host before the introduction of the first chimeric gene.

In Method 2 of the present invention, the first chimeric gene is preferably expressed under the control of an inducible promoter. Thus, the recombinant cell host that has been transformed with the three chimeric genes first expresses constitutively the second and the third chimeric gene in order to allow the interaction of the resulting second and third fusion polypeptides to take place. Then the expression of the first chimeric gene is induced using the appropriate inducing signal, such as the addition of an inducer molecule in the culture medium. For example, the inducible promoter Met 3E (inducible by the amino acid methionine)²⁹ may be used to control the expression of the first chimeric gene.

For the purpose of describing this invention, a gene or a chimeric gene means a polynucleotide that encodes a polypeptide or a fusion polypeptide respectively, wherein the polynucleotide may or may not additionally include a polynucleotide sequence that drives its expression at the transcriptional or translational level.

In a preferred embodiment of the methods of this invention, some of the polynucleotides obtained at step f) or g) of Method 1 or step g) or h) of Method 2 are (simultaneously with completion of the remaining steps in each method with the remaining polynucleotides) subjected to a DNA amplification reaction with a pair of primers, wherein at least one of the primers comprises, at its 5′ end, a promoter region recognized by a specific RNA polymerase (e.g., the bacteriophage T7 promotor region) and then incubated in the presence of the corresponding RNA polymerase, such as the bacteriophage T7 polymerase, in an acellular enzyme medium. The mRNA is then further incubated in the presence of a reverse transcriptase type enzyme and the resulting cDNA molecule is hybridized to a matrix substrate on which has been bound, at known locations, a plurality of sets of oligonucleotides or polynucleotides of predetermined sequence, each bound set of oligonucleotides being able to hybridize with a specific polynucleotide carried by the genome of the organism from which the polynucleotide coding for the first test polypeptide belongs. The polynucleotide hybrid complexes obtained on the matrix substrate are then detected and compared with the results obtained from the matrix of Method 1 or Method 2.

It will be noted in the practice of the methods of this invention, that the polynucleotide inserts of the DNA library used to make the two-hybrid screening step may begin with a nucleotide which is not in phase with the transcriptional activation domain coding sequences. Despite the open reading frame shift occurring at the 5′ end of the polynucleotide sequence, it has been observed that a correct polypeptide is synthesized, due to a probable jump of the ribosome, placing the ribosome back in the correct reading frame. Consequently, a shift in the reading frame at the beginning of the coding sequence of interest does not prevent the synthesis of the correct polypeptide interactor.

In a most preferred embodiment of the methods according to this invention, the selected polynucleotides encoding the first polypeptide are labeled before performing the hybridization step, either during or after the PCR amplification step. The polynucleotide may be labeled with a radioactive element (³²P, ³⁵S, ³H, ¹²⁵I) or by a non-isotopic molecule (for example, biotin, acetylaminofluorene, digoxigenin, 5-bromodesoxyuridin, fluorescein). Examples of non-radioactive labeling of nucleic acid fragments are described in French Patent No. 7810975 or Uredea, or Sanchez-Pescador et al.^{30,31} One of skill in the art will appreciate that other labeling techniques may also be used, such as those described in French Patent Nos. 2422956 and 2528755 or in Matthews et al.³²

One of the most important features of the hybridized DNA arrays or matrices utilized in the screening methods of this invention is that the DNA arrays allow, in a one step method, mapping of all the potential polypeptides interacting with a given defined polypeptide in a forward two-hybrid method, or inhibiting the interaction between two defined polypeptides in a reverse two hybrid method. Thus, the hybridization pattern of oligo- or polynucleotides coding for the interactor polypeptides identify the whole set of polypeptides of interest. In contrast, the prior art technique of systematic sequencing of every selected polynucleotide identified only individual interactor coding sequences and did not provide any understanding of the global interaction possibilities.

Preferably, the oligonucleotide or polynucleotide probes bound to the substrate matrix in the methods of this invention are designed in such a manner that every region of the whole genome of the prokaryotic or eukaryotic host organism is able to specifically hybridize to at least one set of the oligonucleotide or polynucleotide probes. It is also preferred that sets of oligonucleotide or polynucleotide probes bound to the matrix substrate are complementary to adjacent sequences in the genome of the prokaryotic or eukaryotic host, such that the distance between the sequences is less than 500 nucleotides and most preferably about 50 nucleotides.

It will also be apparent that the matrices obtained from the methods of this invention are valuable products themselves. Of particular interest is a matrix substrate comprising a plurality of immobilized sets of oligonucleotide or polynucleotide probes of predetermined sequence, each bound set of oligonucleotide or polynucleotide probes being able to hybridize with a specific polynucleotide carried by the genome of the organism from which the polynucleotide coding for the first test polypeptide belongs; and at least one polynucleotide coding for one selected first test polypeptide being hybridized thereto.

The DNA arrays used in the methods of the invention preferably contain oligonucleotide probes of between 10 and 100 nucleotides, and preferably between 10 and 40 nucleotides, and cover the whole genome or part of the genome of interest. In one embodiment of the invention, the oligonucleotide probes immobilized onto the substrate matrix consist of Expressed Sequence Tags (ESTs). The DNA arrays of this invention may, alternatively, contain full length coding polynucleotides corresponding to every identified gene of the host organism under study. For example, when S. cerevisea is the target host, a typical DNA array used in performing the screening methods of the invention may contain 6000 full length polynucleotides, each polynucleotide comprising the full length coding sequence of a gene among the 6000 genes identified for S. cerevisiae.

Because the screening methods according to this invention make use of DNA probe arrays in order to identify the selected polynucleotides coding for the interactor polypeptides of interest, the methods are particularly well suited to polynucleotides derived from a host organism for which the whole genome has already been sequenced. However, the methods of this invention may also be applied to polynucleotides issued from a library generated from specific partially or totally sequenced chromosomes of complex host organisms, including humans. In one specific embodiment of the methods of this invention, the method is performed using, as a source of polynucleotide sequences to be tested, a library of randomly synthesized and identified polynucleotides.

It will be readily apparent to those of skill in the art that application of the methods of this invention will lead to the identification of novel polynucleotides and their functions. These polynucleotides and the polypeptides encoded by these polynucleotides are within the scope of this invention. Of particular interest are peptides comprising a peptide domain that interacts with the second test polypeptide of interest.

### EXAMPLES

Preparation of Oligonucleotide Arrays

Oligonucleotide arrays containing over 65,000 DNA synthesis features were prepared using light-directed, solid phase combinatorial chemistry as previously described.^{6,7} Each 50×50 μm synthesis feature is comprised of more than 10⁷ copies of a discrete 25-mer oligonucleotide that is complementary to a portion of a yeast gene. The full set of oligonucleotides includes an average of twenty synthesis features for each of the 6,321 genes identified from the Saccharomyces cerevisiae genome. These arrays were originally designed and used for the analysis of mRNA gene expression (Wodicka, L., Dong, H., Mittmann, M., Ho, M. H., and Lockhart, D. J., manuscript in preparation).

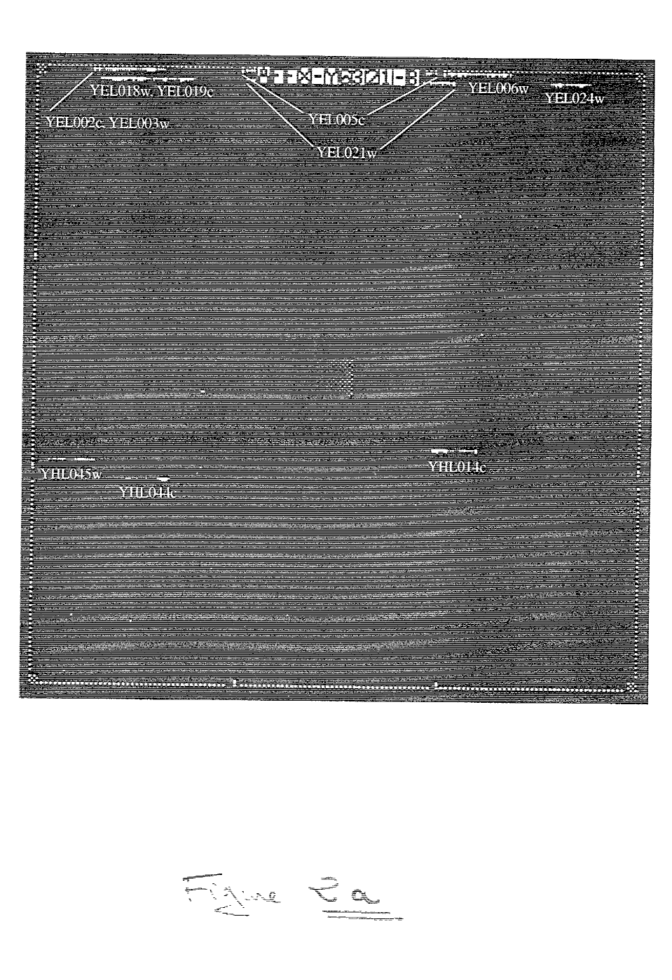

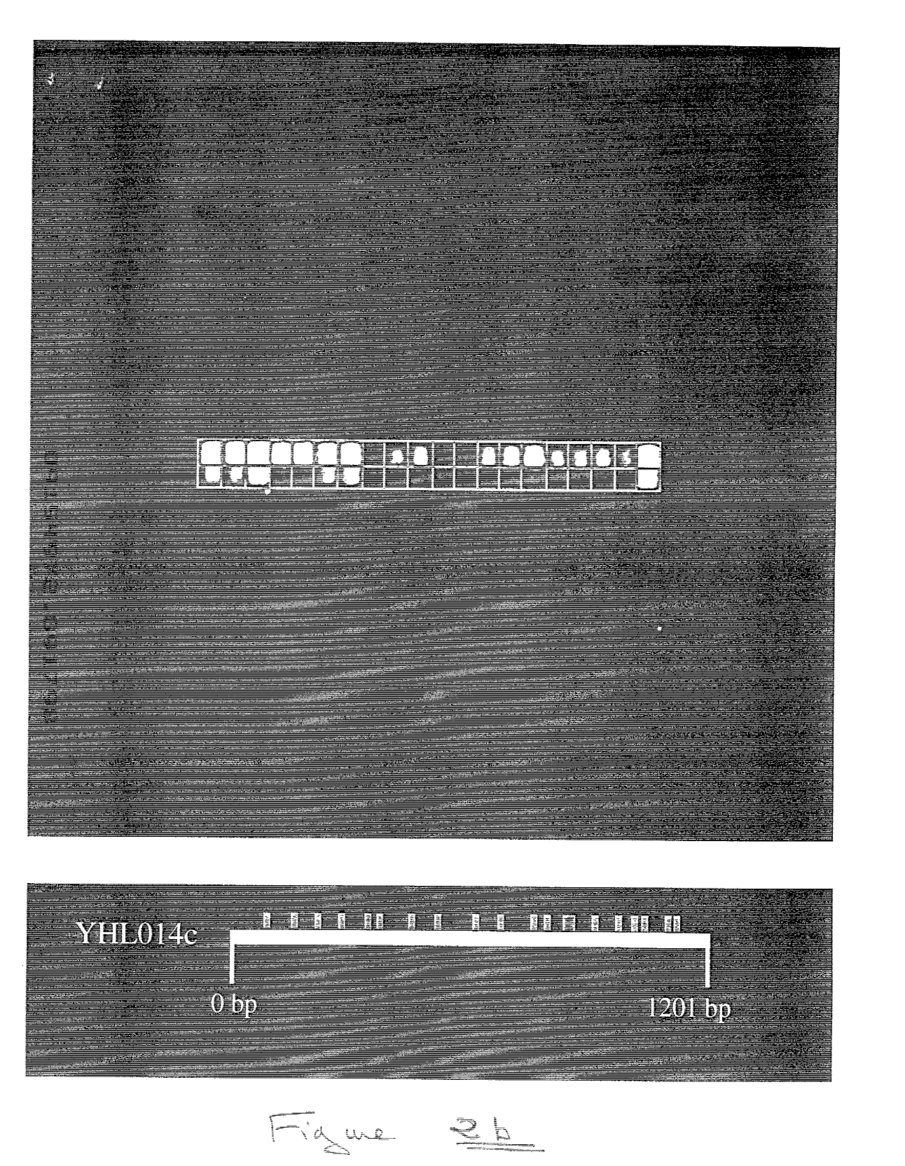

Oligonucleotide arrays were first tested for the ability to identify specific gene fragments. A fluorescence image of an array following hybridization of eleven labeled PCR products reveals intense signals at discrete positions, with minimal background (FIG. 2a). Because the probes for a given gene are synthesized in adjacent positions, hybridization of PCR products is detected as horizontal rows of high intensity (FIG. 2b). Signal corresponding to all eleven genes was detected in the correct locations. No significant signal was detected for any other genes in the genome. Each experiment was performed in duplicate, and hybridization results were found to be reproducible (data not shown).

After a biological selection, library elements in high abundance can be identified by dideoxy sequencing. However, detection of rare elements might require the sequencing of thousands of clones. To determine the ability to detect very rare elements using array hybridization, the control PCR products were remade without the 600 bp YEL006c gene fragment, and known amounts of this sequence were added to the pool. Concentrations of spiked YEL006c DNA as low as 5 pM were detectable by hybridization. Therefore, array hybridization is sensitive to library elements that comprise less than 1:10,000 of the total pool. This is consistent with previous gene expression experiments in which rare mRNAs present at frequencies below 1:100,000 were detected quantitatively.⁷

Whole genome yeast arrays were then used to analyze DNA results from two-hybrid screens for protein-protein interactions. Identification of proteins that physically interact within the cell can suggest how a gene product participates in cellular processes. In the two-hybrid screen, two proteins are expressed in yeast as fusions to either the DNA-binding domain or the activation domain of a transcription factor. Physical interaction of the two proteins reconstitutes transcriptional activity, turning on a chromosomal gene essential for survival under selective conditions.⁸ In screening for novel protein-protein interactions, yeast cells are first transformed with a plasmid encoding a specific DNA-binding fusion protein. A plasmid library of activation domain fusions derived from genomic DNA is then introduced into these cells. Transcriptional activation fusions found in cells which survive selective conditions are considered to encode peptide domains which may interact with the DNA-binding domain fusion protein.

Library Construction

A large yeast genomic DNA library of 5×10⁶ clones (designated the “FRYL” library) was made in E. coli MR32 strain according to a previously described procedure [Elledge et al. PNAS, USA, 88, 1731-1735 (1991)].

Origin of the plasmid: pACTII (with minor modifications).

Origin of the genomic DNA: Ym955 (a gift of M. Johnston).

Ym955=ura3-52, his3-200, ade2-101, lys2-801, leu2-3,112, trp1-901, tyr1-501, gal4-542, gal80-538.

his3-200, trp1-901, gal4-542 and gal80-538 are deletions of all coding sequences.

Genomic DNA was sonicated, blunted by 3 modification enzymes (Mung bean, T4 DNA Polymerase and Kleenow). Adaptors were ligated to blunted ends. Adaptors were designed to allow blunt litigation at one extremity and cohesive ligation with a 3 nucleotide overhang at the other end.

The sequence of adaptors was 5′-ATCCCGGACGAAGGCC (SEQ ID NO: 1) and 5′-GGCCTTCGTCCGG (SEQ ID NO: 2), and only the former was phosphorylated before annealing to avoid self-ligation of the adaptors. After ligation the inserts were purified from free adaptors and small fragments on a Chroma Spin column (Clontech).

The pACTII vector was digested with BamHI and the extremities were filled in with dGTP by the Vent (exo⁻) polymerase (New England Biolabs), generating extremities complementary to the 3 nucleotide overhang of adaptors but preventing self-ligation of the vector. (BamHI sites are reconstituted at each end of the insert). This strategy prevents self-ligation of the vector or ligation of multiple inserts.

Inserts and vectors were ligated together and ligation products were used to transform E. coli MR32. 5×10⁶ clones were obtained. All transformants were scraped from dishes and the pool of transformants were frozen in LB/glycerol. The titer of the library was 1-2×10⁹ transformants/ml.

### Example 1

To demonstrate the analysis of a genetic selection using oligonucleotide arrays, a two-hybrid screen was conducted for the Saccharomyces cerevisiae gene YMR117c. YMR117c is a previously uncharacterized ORF recently found by two-hybrid analysis to interact with the U2 snRNP-associated splicing factor, Prp11p.⁴

Plasmids and Strains

For the YMR117c screen, the yeast strains used for two-hybrid screening were CG1945 and Y187 (Clontech). A pAS2ΔΔ bait vector was constructed from the pAS2 plasmid (Clontech) by deletion of the CYH2 gene and the HA epitope. A bait plasmid was constructed by PCR amplification of YMR117c from genomic DNA and cloning into pAS2ΔΔ as a BamHI-Pst fragment. The bait plasmid was verified by sequencing after cloning.

The polynucleotide insert containing the chimeric gene GAL4/YMR117c consists of SEQ ID NO: 3, wherein nucleotides 1-475 correspond to the GAL4 DNA binding domain. The resulting encoded fusion polypeptide consists of SEQ ID NO: 4, wherein amino acids 1-164 correspond to the GAL4 DNA binding domain and amino acids 165-378 correspond to the YMR117c peptide sequence.

YMR117c Two-hybrid Screen

CG1945 yeast cells were transformed with the bait vector and used in a mating strategy.⁴ Y187 cells were first transformed with DNA from the FRYL two-hybrid library, transformants were pooled, and aliquots of the cell suspension were frozen. The two strains were mixed, concentrated onto filters, and incubated on rich medium for 4.5 h at 30° C. The cells were collected, and a 10⁻³ dilution was spread on -L, -LW, and -W plates to score the number of parental cells and the number of diploids. The rest of the cell suspension was spread on -LWH plates and incubated for three days at 30° C. 8.5×10⁷ diploids were screened, and 5800 His⁺ colonies were selected. 10 ml of an X-Gal mixture (0.5% agar, 0.1% SDS, 6% dimethylformamide, and 0.04% X-Gal) was poured on the plates and the plates were incubated at 30° C. Blue clones were checked after a 30 min to 18 h incubation and streaked on -LWH selective plates. 108 total clones were identified as positive by the X-Gal assay and processed as described below.

PCR Amplification and Labeling of DNA from Pooled Clones

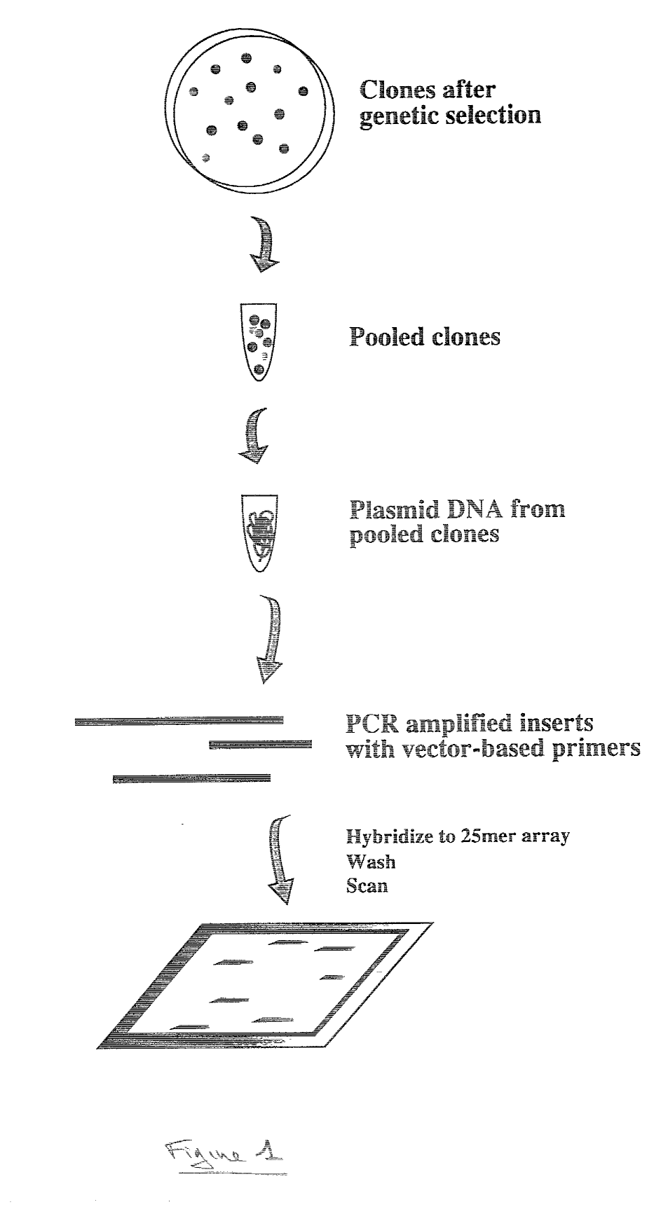

A volume of 200 μl of a saturated culture (approximately 1×10⁷ cells) of each of the 108 positive two-hybrid clones from the YMR117c two-hybrid screen were pooled (FIG. 1) and DNA was isolated and purified as previously described.⁵ Primers containing vector sequence at the 3′ end were used to PCR amplify gene inserts from the plasmid mixture. Specifically, using the vector-based primers T7FOR (5′GAATTGTAATACGA CTCACTATAGGGAGGTGATGAAG ATACCCCACC-3′) (SEQ ID NO: 5) and T3REV (AGATGCAATTAACCCTCACTAAAGGG AGACGGGGTTTTTCAGTATCTAC GATTC-3′) (SEQ ID NO: 6), all library inserts were PCR amplified in a single reaction. The 50 μl PCR reaction contained: 2.5 U of Taq DNA polymerase, 10 mM Tris (pH 8.5), 50 mM KCl, 1.5 mM MgCl₂, 0.2 μM each primer, and 250 μM each dNTP. Conditions used for amplification were as follows: 30 cycles at 96° C. for 30 s, 62° C. for 30 s, 72° C. for 2 min. Reaction products were purified in a Qiaquick spin column (Qiagen). 1 μg total PCR product was fragmented with 0.1 U DNAse I (amplification grade, GibcoBRL) for 2 min in 35 μl containing: 10 mM Tris-acetate (pH 7.5), 10 mM magnesium acetate, 50 mM potassium acetate, and 15 mM CoCl. The DNAse I reaction was then boiled for 15 min, chilled on ice, and incubated with 1 mmole biotin-ddATP (NEN) and 25 U terminal transferase (Boehringer Mannheim) for 1 hour at 37° C. SSPE-T hybridization buffer (0.9 M NaCl, 60 mM NaH₂PO₄, 6 mM EDTA, 0.005% Triton-X-100) was added to a final volume of 200 μl.

Generation of cDNA Product from PCR Product

RNA was transcribed from 240 ng of purified PCR product using T7 polymerase (Ambion). The reaction was incubated an additional hour with 20 U DNAse I. RNA was purified using an RNA spin column (Qiagen). 2.0 μg of RNA was used for first strand cDNA synthesis (Promega). Reaction products were purified in a Qiaquick spin column (Qiagen), and 1 μg total PCR product was digested and prepared for hybridization as described above.

Hybridization of DNA to the High-density Oligonucleotide Array

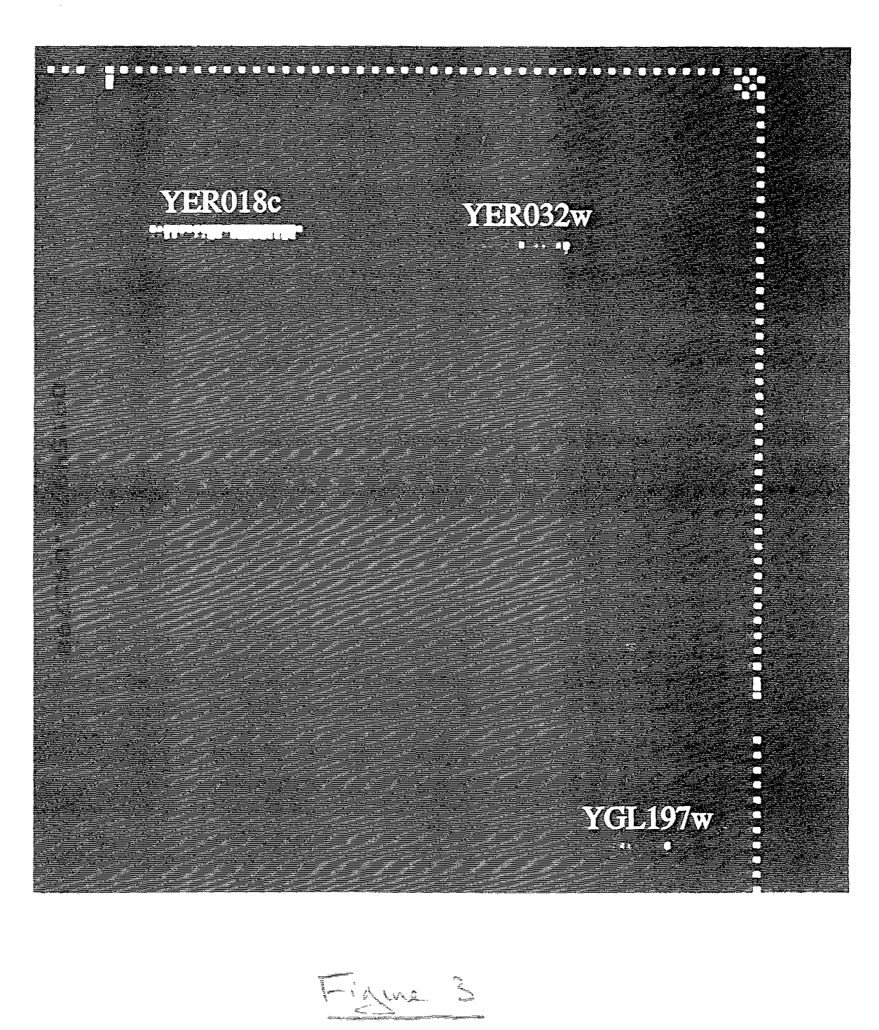

DNA products generated from the library plasmid pool were partially DNAse I digested, biotinylated, and hybridized to whole genome arrays (FIG. 3). Specifically, arrays were prewashed with hybridization buffer (described above) 5 min prior to sample hybridization. Following a 5 min incubation at 99° C., the sample was chilled on ice, allowed to return to room temperature, and applied to the array. After a 12 hour hybridization at 42° C., the array was washed 10 times with 6X SSPE-T, washed with 0.5× SSPE-T for 15 min, and stained with a streptavidin-phycoerythrin conjugate (Molecular Probes) for 10 min, all at 42° C. The staining buffer contained 6X SSPET, 0.5 mg/ml bovine serum albumin, and 1 mg/ml streptavidin-phycoerythrin. The array was washed 5 times with 6X SSPE-T prior to scanning. Hybridization patterns were detected by using an argon ion laser to excite phycoerythrin; the resulting emission was detected using a photomultiplier tube through a 560 nm bandpass filter (Molecular Dynamics). The entire array was read at a resolution of 7.5 μm in less than 20 min, generating quantitative signal for each probe element. The collected data was analyzed using image and data analysis software (Affymetrix).

Orientation of genes was determined by hybridization of biotinylated cDNA products.

All genes identified by array hybridization are listed in Table 1.

Criteria for Gene Detection

On chips A, B, C, and D, which contain an average of 20 probes per gene, the presence of a gene fragment was determined by visual and quantitative detection of three contiguous positive probes. On the E chip, which contains probes for 5′ sequence from genes which are longer than 1 kb, detection of two contiguous positive probes was considered sufficient to detect a gene fragment.

Comparison of Hybridization and Sequencing Results

Library plasmid inserts were amplified by PCR and the insert junctions with the GAL4 domain were sequenced and precisely identified in the yeast genome using the BLAST program, the Saccharomyces Genome Database, and the Yeast Protein Database. In parallel, clones were used to inoculate 200 μl cultures. Saturated cultures were pooled and processed as previously described.

The hybridization results from the YMR117C screen were compared to results obtained by dideoxy sequencing of all 108 DNA clones. Nineteen of twenty-two independent loci were identified by hybridization, with no false positives. Based on analysis of the hybridizing array elements, we were also able to identify the region of the gene present in each insert (Table 2).

The three loci that were not detected by array hybridization were either not represented on the array or were resistant to PCR amplification. One of the undetected inserts, YLR276c, was difficult to amplify by PCR and could only be sequenced after plasmid rescue. The other two undetected inserts start within two hundred bases upstream of the 3′ end of the gene, in region only covered by one or no probes. Therefore, the signal for these genes was not recognized as significant because there was not a consistent pattern of hybridization extending across multiple probes.

### Example 2

To further demonstrate this method, a two-hybrid screen for the gene YMR138w was also carried out and analyzed by array hybridization. YMR138w (CIN4) is a gene in which mutations cause supersensitivity to the antimicrotubule drug benomyl, as well as increased rates of chromosome loss.¹² YMR138w is homologous to the ARF1-class of small GTP-binding proteins, but a distinct role in microtubule function is not yet known. The complete results for this screen are listed in Table 1.

Plasmids and Strains

For the YMR138w screen, the yeast strains used were the Y190 and Y187 cyh2^{R} marked derivatives of Y159 and Y153, respectively. The library was a yeast cDNA library fused to the transcriptional activation domain of GAL4 (gift of S. Elledge, Baylor College of Medicine). The bait vector pTS434 was constructed by cloning CIN4 into pAS1-CYH2 (Clontech) as a NcoI-BamHI fragment.

YMR138W Two-hybrid Screen

Y190 containing pTS434 was transformed with cDNA library using a lithium acetate-based protocol. 5×10⁶ transformants were screened by plating on −Ade selective media, and 114 colonies Ade⁺ were selected. All 114 colonies were patched onto +Ade plates and lifted onto BA85 nitrocellulose filters (Schleicher and Schuell) and immersed in liquid nitrogen for 10 s. The filters were then soaked with 3 mls of Z buffer (60 mM Na2HPO₄, 40 mM NaH2PO₄, 10 mM KCl, 1mM MgSO₄, and 50 mM β-mercaptoethanol; pH 7.0) containing 0.05% X-Gal. Filters were incubated at 30° C. for 6 h and scored for the development of blue color. 86 clones were positive by a lacZ filter assay. All 86 clones passed testing for solo activation by streaking strain Y190 carrying the library isolate and pTS434 on -L plates plus 5 μg/ml cycloheximide. The strains were confirmed to have lost the TRP-containing plasmid by failure to grow on -W media. 81 clones passed testing for specificity by mating strain Y190 carrying library plasmids with Y187 carrying the negative controls pAS-CDK2, pAS10-lamin, pAS1-p53, and pAS1-rev (a gift of D. Amberg). Library plasmid inserts were amplified by PCR and the insert junctions with the GAL4 domain were sequenced and precisely identified in the yeast genome using the BLAST program, the Saccharomyces Genome Database (http://genome-www.stanford.edu) and the Yeast Protein Database (http://www.proteome.com). In parallel, clones were used to inoculate 200 μl cultures. Saturated cultures were collected, pooled, and processed as previously described.

Hybridization of DNA to the High-density Oligonucleotide Array

DNA products generated from the library plasmid pool were partially DNAse I digested, biotinylated, and hybridized to whole genome arrays. Specifically, arrays were prewashed with hybridization buffer (described above) 5 min prior to sample hybridization. Following a 5 min incubation at 99° C., the sample was chilled on ice, allowed to return to room temperature, and applied to the array. After a 12 hour hybridization at 42° C., the array was washed 10 times with 6x SSPE-T, washed with 0.5× SSPE-T for 15 min, and stained with a streptavidin-phycoerythrin l conjugate (Molecular Probes) for 10 min, all at 42° C. The staining buffer contained 6× SSPET, 0.5 mg/ml bovine serum albumin, and 1 mg/ml streptavidin-phycoerythrin. The array was washed 5 times with 6× SSPE-T prior to scanning. Hybridization patterns were detected by using an argon ion laser to excite phycoerythrin; the resulting emission was detected using a photomultiplier tube through a 560 nm bandpass filter (Molecular Dynamics). The entire array was read at a resolution of 7.5 μm in less than 20 min, generating quantitative signal for each probe element. The collected data was analyzed using image and data analysis software (Affymetrix).

Orientation of genes was determined by hybridization of biotinylated cDNA products. All genes identified by array hybridization are listed in Table 1.

Conclusion

Both two-hybrid screens identified interactors consistent with known results for each gene. The previously detected interaction of YMR117c with Prp11p splicing factor has suggested that YMR117c could have a functional connection with the U2snRNP.⁴ Several of the interactors found in this screen also have known associations with the U2snRNP. For example, Ym1049c has previously been found to interact with the Prp9p splicing factor.⁴ Like CIN4, YPL241c (CIN2) was first isolated as a mutation displaying supersensitivity to antimicrotubule agents.¹² Mutations in both CIN2 and CIN4 have already been shown to be epistatic to mutations in CIN1, a gene implicated in the post-chaperonin folding of yeast tubulin.¹³ However, these results are the first evidence for a physical interaction between CIN2 and CIN4 and suggest that they may act as a complex to regulate specific protein-folding pathways. Further investigations are needed to establish the biological significance of interactions from both screens.

TABLE 1
Yeast ORFs identified by array analysis of two-hybrid screens

| | YMR117c | YMR138w (CIN4) |
|---|---|---|
| | YBR020w (GAL1) | YDL117w |
| | YCL032w (STE50) | YDR087c |
| | YCR073c (SSK22) | YGL172w (NUP49) |
| | YDR104c | YHR141c (MAK18) |
| | YER018c | YLR109w |
| | YER032w (FIR1) | YNR050c (LYS9) |
| | YFR046c | YPL241c (CIN2) |
| | YGL197w | |
| | YIL144w | |
| | YLR319c (BUD 6) | |
| | YLR419w | |
| | YML049c | |
| | YMR224c (MRE11) | |
| | YOL18c | |
| | YOL34w | |
| | YOR206w | |
| | YPR010c (RPA135) | |
| | YPR145w (ASN1) | |
| Non-protein | | 18s and 25s rRNA |
| encoding DNA | | |
| Reverse | YNL291c | YBR189w |
| orientation | | YDR381w |
| | | YNL301c (RP28B) |
| | | YNR035c |
| | | YOL056w (GPM3) |
| ORF loci and names are listed for genes detected by array hybridization of PCR products derived from end-products of a two-hybrid screen. Because inserts in the non-coding orientation comprise a significant proportion of false positives in the two-hybrid screen, RNA was transcribed from the upstream T7 promoter and used to generate exclusively antisense cDNA strands with reverse transcriptase. cDNA products were then biotinylated, fragmented, | | |
| #and hybridized as described. Genes detected by double stranded DNA hybridization but absent in cDNA hybridization are considered to be in reverse orientation. Control experiments were performed to confirm that this method is orientation-specific (data not shown). | | |

TABLE 2
Comparison of sequencing and hybridization for clone 5′ ends

| ORF name | ORF size (nt) | 5′ end by sequencing | 5′ end array probe |
|---|---|---|---|
| YBR020w | 1584 | 1151 | 1164 |
| YCL032w | 1038 | 131 | 168 |
| YDR104c | 3735 | 3230 | 3234 |
| YER032w | 2775 | 1808 | 1860 |
| YFR046c | 1083 | 4 | 114 |
| YGL197w | 4461 | 3974 | 4092 |
| YML049c | 4083 | 2597 | 2616 |
| YMR224c | 2076 | 531 | 566 |
| YOL018c | 1191 | 257 | 324 |
| YOL034w | 3279 | 620 | 669 |
| ORF name, ORF size, and the 5′ ends of identified genes, determined either by sequencing or array hybridization, for 10 clones from the YMR117c screen. For genes sequenced multiple times as different inserts, the end of the most 5′ clone is listed. The 5′ end as detected by array hybridization indicates the most 5′ nucleotide of the most 5′ probe detected as positive. Small disparities between sequencing and hybridization are | | | |
| #the result of insert 5′ ends falling in between probes on the array. Although array hybridization does not confirm that inserts are in frame with respect to the start codon, previous work has shown that frameshifting events generally lead to production of protein regardless of the precise fusion junction between gene insert and transcriptional activation domain.¹¹ | | | |

### REFERENCES

1. Goffeau, A. et al. (1996) Science. 274, 546, 563-7.

2. Oliver, S. G. (1996) Nature. 379, 597-600.

3. Fields, S. (1997) Nat Genet. 15, 325-327.

4. Fromont-Racine, M., Rain, J. C. & Legrain, P. (1997) Nat Genet. 16, 277-282.

5. Hoffinan, C. S. & Winston, F. (1987) Gene. 57, 267-272.

6. Chee, M. et al. (1996) Science. 274, 610-4.

7. Lockhart, D. J. et al. (1996) Nature Biotechnology. 14, 1675-1680.

8. Fields, S. & Stemglanz, R. (1994) Trends Genet. 10, 286-92.

9. Hollenberg, S. M., Stemglanz, R., Cheng, P. F. & Weintraub, H. (1995) Mol and Cell Bio. 15, 3813-3822.

10. Mendelsohn, A. R. & Brent, R. (1994) Curr Opin in Biotech. 5, 482-486.

11. Harper, J. W., Adami, G. R., Wei, N., Keyomarsi, K. & Elledge, S. J. (1993) Cell. 75,805-816.

12. Steams, T., Hoyt, M. A. & Botstein, D. (1990) Genetics. 124, 251-262.

13. Stearns, T. (1988) Massachusetts Institute of Technology.

14. Lander, E. S. (1996) Science. 274, 536-9.

15. Shoemaker, D. D., Lashkari, D. A., Morris, D., Mittmann, M. & Davis, R. W. (1996) Nat Genet. 14, 450-6.

16. Smith, V., Chou, K. N., Lashkari, D., Botstein, D. & Brown, P. O. (1996) Science. 274, 2069-74.

17. Klein, R. D., Gu, Q., Goddard, A. & Rosenthal, A. (1996) Proc Natl Acad Sci US A. 93, 7108-13.

18. Kroll, E. S., Hyland, K. M., Hieter, P. & Li, J. J. (1996) Genetics. 143, 95-102.

19. Bartel, P. L., Roecklein, J. A., SenGupta, D. & Fields, S. (1996) Nat Genet. 12, 72-7.

20. Amberg, D. C., Basart, E. & Botstein, D. (1995) Nat Struct Biol. 2, 28-35.

21. Fields, S. & Song, O. (1989) Nature 340, 245-6; Chien, C. T., Bartel, P. L., Sternglanz, R. & Fields, S. (1991) Proc. Natl. Acad. Sci. USA. 88, 9578-82.

22. Rossi, F., Charlton, C. A. & Blau, H. M. (1997) Proc. Natl. Acad. Sci. USA. 94, 8405-8410.

23. Ullmann, A., Jacob, F. & Monod, J. (1968) J. Mol. Biol. 32, 1-13.

24. Smith, G. P. (1985) Science 228, 1315-7; Scott, J. K. & Smith, G. P. (1990) Science 249, 386-90.

25. Gernino, F. J., Wang, Z. X. & Weissman, S. M. (1993) Proc. Natl. Acad. Sci. USA. 90, 933-7.

26. White (1996) Proc. Natl. Acad. Sci. USA. 93, 10001-10003.

27. Vidal et al. (1996) Proc. Natl. Acad. Sci. USA. 93, 10315-10320.

28. Vidal et al. (1996) Proc. Natl. Acad. Sci. USA. 93, 10321-10326.

29. Cherost et al. (1985) Gene, 34, 269-281.

30. Uredea, M. S. (1988) Nucleic Acid Research, 11, 4937-4957.

31. Sanchez-Pescador, R. et al. (1988) J. Clin. Microbiol., 26(10), 1934-1938.

32. Matthews, J. A. et al. (1989) Anal. Biochem., 169, 1-25.

What is claimed is:

1. A method for identification of a polynucleotide comprising the steps of:
a) subjecting a polynucleotide of interest to a two-hybrid screening method;
b) subjecting the polynucleotides selected at step a) to a hybridization reaction onto a matrix substrate onto which oligonucleotide or polynucleotide probes have been immobilized.

2. A method for identifying a polynucleotide encoding a first polypeptide, said first polypeptide being able to interact with a second polypeptide of interest, comprising the steps of:
a) providing a recombinant host cell containing a detectable gene, wherein the detectable gene expresses a detectable polypeptide when the detectable gene is activated by an amino acid sequence including a transcriptional activation domain;
b) providing a first chimeric gene that is capable of being expressed in the host cell, the first chimeric gene comprising a DNA sequence that encodes a first hybrid polypeptide encoded by a given prokaryotic or eukaryotic organism, said first hybrid polypeptide comprising:
(i) the transcriptional activation domain; and
(ii) a first test polypeptide that is to be tested for interaction with the second test polypeptide;
c) providing a second chimeric gene that is capable of being expressed in the host cell, the second chimeric gene comprising a DNA sequence that encodes a second hybrid polypeptide, the second hybrid polypeptide comprising:
(i) a DNA-binding domain that recognizes a binding site on the detectable gene in the host cell; and
(ii) a second test polypeptide that is to be tested for interaction with at least one first test polypeptide;
wherein interaction between the first test polypeptide and the second test polypeptide in the host cell causes the transcriptional activation domain to activate transcription of the detectable gene;
d) introducing the first chimeric gene and the second chimeric gene into the host cell;
e) subjecting the host cell to conditions under which the first hybrid polypeptide and the second hybrid polypeptide are expressed in sufficient quantity for the detectable gene to be activated;
f) selecting the host cell clones for which the detectable gene has been expressed to a degree greater than expression in the absence of interaction between the first test polypeptide and the second test polypeptide;
g) optionally pooling the clones that have been positively selected at step f)
h) amplifying the polynucleotides of interest contained in the clones of step f) or g) with a pair of oligonucleotide primers respectively hybridizing with a plasmid sequence located at the 5′ end of the polynucleotide of interest and with a sequence complementary to a plasmid sequence located at the 3′ end of the polynucleotide of interest coding for the first polypeptide;
i) hybridizing the amplified polynucleotides obtained at step h) to a matrix substrate on which has been bound, at known locations, a plurality of sets of oligonucleotide or polynucleotide probes of predetermined sequence, each bound set of oligonucleotide or polynucleotide probes being able to hybridize with a specific polynucleotide carried by the genome of the organism from which the polynucleotide coding for the first test polypeptide belongs;
j) detecting the locations of the polynucleotide hybrid complexes obtained at step i) on the matrix substrate;
k) optionally determining the quantity of each hybrid complex detected at step j).

3. A method for identifying a polynucleotide encoding a first polypeptide that inhibits the interaction between a second polypeptide and a third polypeptide comprising the steps of:
a) providing a recombinant host cell containing a detectable gene wherein the detectable gene expresses a detectable polypeptide when the detectable gene is activated by an amino acid sequence including a transcriptional activation domain;
b) providing a first gene that is capable of being expressed in the host cell, said first gene comprising a DNA sequence that encodes a first polypeptide encoded by a given prokaryotic or eukaryotic organism, and for which its inhibition properties on the interaction between a second and a third polypeptide is tested;
c) providing a second chimeric gene that is capable of being expressed in host cell, the second chimeric gene comprising a DNA sequence that encodes a second hybrid polypeptide encoded by a given prokaryotic or eukaryotic organism, said second hybrid polypeptide comprising:
(i) the transcriptional activation domain; and
(ii) a second test polypeptide that interacts with a third polypeptide;
d) providing a third chimeric gene that is capable of being expressed in the host cell, the third chimeric gene comprising a DNA sequence that encodes a third hybrid polypeptide, the third hybrid polypeptide comprising:
(i) a DNA-binding domain that recognizes a binding site on the detectable gene in the host cell; and
(ii) a third test polypeptide that interacts with the second test polypeptide;
wherein interaction between the second test polypeptide and the third test polypeptide in the host cell causes the transcriptional activation domain to activate transcription of the detectable gene;
e) introducing the first gene, the second chimeric gene and the third chimeric gene into the host cell;
f) subjecting the host cell to conditions under which the second hybrid polypeptide and the third polypeptide are expressed in sufficient quantity for the detectable gene to be activated;
g) selecting the host cell clones for which the detectable gene has been expressed to a degree lesser than its expression level in the absence of expression of the first polypeptide;
h) optionally pooling the clones that have been positively selected at step g)
i) amplifying the polynucleotides of interest contained in the clones of step g) or h) with a pair of oligonucleotide primers respectively hybridizing with a plasmid sequence located at the 5′ end of the polynucleotide of interest and with a sequence complementary to a plasmid sequence located at the 3′ end of the polynucleotide of interest coding for the first polypeptide.
j) hybridizing the amplified polynucleotides obtained at step i) to a matrix substrate on which has been bound, at known locations, a plurality of sets of oligonucleotide or polynucleotide probes of predetermined sequence, each bound set of oligonucleotide or polynucleotide probes being able to hybridize with a specific polynucleotide carried by the genome of the organism from which the polynucleotide coding for the first test polypeptide belongs;
k) detecting the locations of the polynucleotide hybrid complexes obtained at step j) on the matrix substrate;
l) optionally determining the quantity of each hybrid complex detected at step i).

4. The method according to claim 2 or 3, wherein
a) some of the polynucleotides obtained at step f) or g) of claim 2 or at step g) or h) of claim 3 are separated and subjected to a DNA amplification reaction with a pair of primers wherein at least one of the primers comprises, at its 5′ end, a promoter region recognized by a specific RNA polymerase;
b) the resulting amplified polynucleotides of the above step a) are incubated in the presence of the corresponding RNA polymerase in an acellular enzyme medium;
c) the mRNA obtained at the above step b) is incubated in the presence of a reverse transcriptase type enzyme;
d) the cDNA molecule obtained at the above step c) is hybridized to a matrix substrate on which has been bound, at known locations, a plurality of sets of oligonucleotides of predetermined sequence, each bound set of oligonucleotide being able to hybridize with a specific polynucleotide carried by the genome of the organism from which the polynucleotide coding for the first test polypeptide belongs; and
e) the locations of the polynucleotide hybrid complexes obtained at step d) on the matrix substrate are determined and compared with the results obtained from method of claim 2 or claim 3.

5. The method according to any one of claims 1 to 3, wherein the transcriptional activator is from GAL4.

6. The method according to any one of claims 1 to 3, wherein the transcriptional activator is from GAL4.

7. The method according to claim 4, wherein the promoter region contained in the primer used at step a) is the bacteriophage T7 promoter region and the RNA polymerase used at step b) is the bacteriophage T7 polymerase.

8. The method according to any one of claims 1 to 3 wherein the part of the first chimeric gene coding for the first test polypeptide is provided by a DNA library.

9. The method according to claim 8 wherein the DNA library has been prepared from the genome or from the mRNA of a prokaryotic host.

10. The method according to claim 8 wherein the DNA library has been prepared from the genome or from the mRNA of a eukaryotic host.

11. The method according to claim 10 wherein the DNA library has been prepared from the genomic DNA of Saccharomyces cerevisiae.

12. The method according to any one of claims 1 to 3, wherein the sets of oligonucleotide or polynucleotide probes bound to the substrate matrix are designed in such a manner that every region of the whole genome of the prokaryotic or eukaryotic host organism is able to specifically hybridize to at least one of said set of oligonucleotide or polynucleotide probes.

13. The method according to claim 12, wherein two sets of oligonucleotide or polynucleotide probes bound to the matrix substrate are complementary to adjacent sequences in the genome of the prokaryotic or eukaryotic host distant one from each other of less than one kilobase.

14. The method according to claim 13, wherein two sets of oligonucleotide or polynucleotide probes bound to the matrix substrate are complementary to adjacent sequences in the genome of the host, such that the distance between the sequences is less than 500 bases.

15. The method according to claim 14, wherein two sets of oligonucleotide or polynucleotide probes bound to the matrix substrate are complementary to adjacent sequences in the genome of the host, such that the distance between the sequences is about 50 bases.

16. A polynucleotide molecule that has been obtained with the method according to any one of claims 1 to 3.

17. A polypeptide that is encoded by a polynucleotide according to claim 16.

18. A polypeptide that has been obtained with the method according to any one of claims 1 to 3.

19. A peptide comprising a peptide domain interacting with the second test polypeptide of interest.

20. A matrix substrate on which has been bound, at known locations, a plurality of sets of oligonucleotide or polynucleotide probes of predetermined sequence, each bound set of oligonucleotide or polynucleotide probes being able to hybridize with a specific polynucleotide carried by the genome of the organism from which the polynucleotide coding for the first test polypeptide belongs.

21. A matrix substrate comprising:
a) a plurality of immobilized sets of oligonucleotide or polynucleotide probes of predetermined sequence, each bound set of oligonucleotide or polynucleotide probes being able to hybridize with a specific polynucleotide carried by the genome of the organism from which the polynucleotide coding for the first test polypeptide belongs;
b) at least one polynucleotide coding for one selected first test polypeptide being hybridized thereto.

22. A computer useable medium containing computer readable data related to the hybrid complexes formed within a matrix substrate according to claim 20 or claim 21.
